(19) United States
(12) Patent Application Publication
(10) Pub. No.: US 2022/0226420 A1
(43) Pub. Date: Jul. 21, 2022

(54) HEALTH PRODUCTS FROM GARLIC GREEN TOPS

(71) Applicant: Australian Garlic Producers Pty Ltd, Iraak (AU)

(72) Inventors: Ambrosios KAMBOURIS, Koorlong (AU); Nick DIAMANTOPOULOS, Malvern (AU)

(73) Assignee: Australian Garlic Producers Pty Ltd, Iraak (AU)

(21) Appl. No.: 17/614,764

(22) PCT Filed: May 28, 2020
(86) PCT No.: PCT/AU2020/050539

§ 371 Date: Nov. 29, 2021

(30) Foreign Application Priority Data
May 29, 2019 (AU) 2019901848

(51) Int. Cl. A61K 36/8962; A61K 31/10; C12P 7/08; C12P 11/00; A23L 33/105; A23L 33/115

(52) U.S. Cl. CPC A61K 36/8962; A61K 31/10; C12P 7/08; A23V 2002/00; A23L 33/105; A23L 33/115; C12P 11/00

(57) ABSTRACT

A process for harvesting and preparing green garlic leaves and shoots (green tops) for the production of ajoene and garlic oil for use in medicine and foods, the method including the steps of garlic green top separation from bulbs during harvesting, collecting, shredding, blending, extracting or converting and then adding to fortify butter, margarine and medicinal capsules and edible oils.

### FIELD OF THE INVENTION

The present invention relates to the harvesting and processing of garlic green tops.

### BACKGROUND TO THE INVENTION

Harvesting of the garlic plant commercially involves unearthing the whole garlic plant using specialized equipment that travels beneath the garlic plant and mechanically lifts the plant out from beneath the soil surface.

Such ripe garlic plants are often harvested commercially whilst the leaves and shoots (garlic tops) are still green. In such instances, the green garlic tops (material above the garlic bulb), could represent between 30% and 50% of the weight of the total garlic plant.

Subsequently, the garlic plant is cut to separate the garlic bulb from the garlic tops. Garlic tops whilst green have until now, had no commercial value and have been discarded either at the harvest site or at the packing house. For this reason, equipment has not been designed specifically for collecting such green tops.

Some consumers do use the whole delicate immature garlic plants (bulb, leaf, shoots) in fresh salads and other foods. However, once the garlic plant matures and the bulb increases in size, it is no longer tender to consume the whole plant.

Instead, the large mature garlic bulb alone is used for human consumption, medicine and cooking and for extraction of valuable garlic constituents such as garlic oil, alliin, allicin, ajoene and others. In all such instances, it is preferential for the garlic bulbs to have much of the husks removed.

Garlic bulbs need to be finely macerated after most husks are removed to produce edible garlic paste, or garlic oil, or to produce allicin rich fractions used for converting into ajoene.

Ajoene is an organosulfur compound found in garlic extracts. It is a colourless liquid that contains sulfoxide and disulfide functional groups. The name is derived from “ajo”, the Spanish word for garlic.

Ajoene is an important compound for health benefits such as being a broad-spectrum antimicrobial, antioxidant, antithrombotic, prevention of yeast infection and inhibition of gene-controlled quorum sensing activity.

The Ajoene is very low in concentration in raw garlic bulbs, so this garlic is further processed to convert precursors such as allicin or diallyl disulphide into Ajoene. Such processes are well documented.

Production of Ajoene has been exclusively made from the traditional garlic bulb and often from bulbs that are too ugly or too small to be displayed in supermarkets. Competition between the use of garlic bulbs for health and food use has placed a high price on this commodity, making it very expensive, particularly for the commercial production of Ajoene.

The practice of garlic green top removal from the all green garlic plant at harvest, results in losses of some valuable garlic constituents such as alliin, allicin, Ajoene, garlic oil and other highly valued garlic constituents including sugar.

It is the scope here, to teach of using the entirety of the mature garlic plant or the garlic tops only, for commercial production of garlic oil, allicin, ajoene, alcohol and garlic derived vinegar. This optimizes the value of the garlic plant by allowing greater recovery of active ingredients for commercial use.

The growing of garlic commercially has evolved with technology over time. Plants are propagated from nurseries growing virus free seeds. Garlic varieties are selected according to market requirements and harvesting has become mechanised. However, the removal of the garlic tops commercially remains tradition, except that their removal can be carried out mechanically during harvesting on the field. The reason why the garlic tops are not also used commercially is that growers of garlic have not been aware of nutrient reserves within this portion of the garlic plant.

The object of this invention is to provide a method of harvesting garlic green tops to take advantage of a resource previously discarded and optimise the value of garlic plants.

### SUMMARY OF THE INVENTION

In a first aspect the invention provides a method of producing ajoene and garlic oil products from garlic green tops comprising:

harvesting garlic plants and cutting the green tops from garlic bulbs at the time of harvesting;

collecting the green tops in holding bins; and

transporting the green tops to a processing site wherein the green tops are macerated to produce a green top blend and reduce their transportation volume.

Water may be added to the green top blend to make it flowable.

In preference cell disruptive enzymes are added to the green top blend to release garlic enzymes that convert alliin to allicin and release garlic oil.

Preferably distillation or supercritical CO2 is used to separate the garlic oil from the green top blend and leave spent green tops, and liquid is extracted from the spent green tops and fermented to produce alcohol and even vinegar.

Preferably allicin is extracted from the green top blend and converted into ajoene.

It should be noted that any one of the aspects mentioned above may include any of the features of any of the other aspects mentioned above and may include any of the features of any of the embodiments described below as appropriate.

### DETAILED DESCRIPTION OF THE INVENTION

The present invention provides a method of harvesting garlic green tops to take advantage of a resource previously discarded and optimise the value of garlic plants.

Garlic tops which consist of green garlic leaves and shoots are rich in similar constituents found in the garlic bulb whilst the garlic tops remain green. Such constituents reduce as the garlic tops dry if plants are left in the ground for the bulb to mature fully.

Garlic oil recovered from the leaves and stems of green tops, varies in composition to the garlic oil found in the bulb at the time of harvest. Furthermore, the analytical composition of garlic oil differs when compared to mature (ground dried garlic bulbs) or dried or even aged garlic bulbs of one variety of garlic.

The difference seen between green top and bulb garlic oil is the percentage composition of total sulphur compounds. In the green tops, this amount is always lower than the oil extracted from the bulb of the same garlic plant.

Furthermore, the lower level of such total sulphur compounds is a result of predominantly less diallyl disulphide and diallyl trisulfide. This is biologically significant as each of the different constituents mentioned effects the garlic oil functionality. When the garlic green tops are harvested and isolated from the bulb, it appears that the sulphur compounds are not as developed in secondary and tertiary structure i.e. higher diallyl sulphide form. This may even elicit a more potent biological effect if taken by humans or animals and could be useful for medicinal use.

Modern mechanical garlic harvesting often requires the garlic plant to be green during harvest as it offers the advantage of easy cutting as the whole garlic can remain upright as it travels through cutting saws. Harvesting the garlic plant with green tops results in alliin and garlic oil and fermentable & non-fermentable sugars remaining in the green tops and these are subsequently commercially lost as a result of not using all the garlic plant. In this instance, it reduces grower profit and the productive capacity of the land and other resources. These valuable constituents are simply lost.

Relative to the whole green garlic plant, in some garlic varieties, the shoot contains around 10% less allicin than the average of the whole green garlic plant. The leaf has around 50% less allicin than the average of the whole green plant.

This similar level of garlic oil percentage distribution is also found at optimum maturity from a variety of garlic grown in Australia. The green tops generally have almost 75% w/w garlic oil with respect to w/w garlic oil in the bulb of the same plant.

Furthermore, the distribution of sugar monitored by Brix measurement varied slightly between leaves and shoots but was abundant throughout the plant at harvest.

The garlic bulb rather than the garlic green tops is used for a variety of medicinal and food purposes. The green tops which are traditionally cut off during harvesting and discarded, can whilst still green, provide a large quantity of constituents such as garlic oil, alliin, allicin, ajoene and fermentable sugar. The quantities vary between garlic varieties and climatic conditions but in some instances can be present in levels around 75% of the levels found in the bulb.

Green leaves or stems from the garlic plant contain precursors that can be manipulated by established processes to produce Ajoene and or garlic oil or other desirable products. This unused garlic constituent source also contains constituents or compounds that can economically be extracted directly for either food or health uses. The option to present all such constituents to the health or food market may involve secondary packaging such as capsules or tablets or blisters or bottles or other protective packaging.

Green stems or leaves from garlic plants can be processed as described by patent number: US 2012/0282334 A1, converting the allicin produced during maceration into Ajoene. Alternatively, the sulphur containing compounds such as diallyl disulphide can be converted to Allicin and subsequently to Ajoene by further acidification (AU 2016 203651 A1).

Processing such green stems or leaves has not been taught by prior art and it is the purpose of this embodiment to use any options available to process this starting material to produce similar health and food products that are made from garlic bulbs.

The present invention shows how green garlic tops may be processed intact with immature garlic bulbs to produce valuable constituents whilst not discarding any part of the plant.

A key aspect of the invention is that the green tops are finely macerated during mechanical harvesting to release intracellular constituents and reduce the volume for transportation. Then distilled to remove the garlic oil and the leftover body of the green tops treated to remove solids, diluted and fermented to produce alcohol and oxidised to produce vinegar.

Green tops that have been macerated are optionally enzyme treated to release alliin will be converted to a source of allicin. This allicin source can then be used to produce Ajoene after solids have been removed by a process such as centrifugation, or earth filtering or cross flow filtration or any other means of processing available that convert allicin to Ajoene.

The production of garlic oil from mature garlic bulbs is well known and can be carried out by many traditional processes including the more significant, chemical extraction, distillation and supercritical carbon dioxide extraction processes. Such processes are well described elsewhere. The following disclosure teaches utilising either whole mature garlic plants or the green tops of mature garlic plants alone to ultimately produce garlic constituents that can be commercialised as products for human or animal consumption.

In the present invention garlic tops that are removed during mechanical harvesting can be collected after they are cut from the garlic bulb in the field or in the packing facility. This involves collecting the garlic tops when they are cut from the bulb and conveying them into collection bins. This may involve elevators, to direct the garlic tops from the cutting saws of the harvester. The process may involve sucking or blowing the green tops onto a conveyor system or even mechanically pushing the green tops onto the conveyor rather than dropping them on the ground.

Prior to placing the garlic green tops into transport bins in the field, it is desirable to reduce the volume of the greens to reduce the volume required to transport them for further processing. This may involve a chaff cutter or a crude macerator or even a continuous blender or a combination of such processes. The blending may even require the addition of water to make the blenders effective and to make the blended green tops flowable. It is preferable that the blending used is a continuous blending system carried out on site during harvesting in the field however, the green tops can only be chopped up to reduce their size and to minimise transport volume in the field if required. Continuous blending of the green tops can also then be carried out at the processing site.

At the processing site, these minced or blended green tops can either be frozen, chilled or processed immediately after optional enzyme treatment to break down the cellular vacuoles and fibres to release garlic oil and other constituents from the intracellular environment, including garlic enzymes that convert alliin to allicin.

This unstable allicin in the blended green top mixture can be further converted to Ajoene by several established processes described elsewhere after green top solids are separated from the liquid. Alternatively, the green tops can be directly processed through garlic oil extraction processes such as distillation or other oil extraction processes like chemical.

The slurry is then fed into a spinning cone distillation or other distillation method, including hydro-distillation or microwave assisted microdistillation for garlic oil extraction. Our preferred method of distillation is using the Flavourtech Pty Ltd Australia, Spinning cone distillation plant.

Alternatively, the blended garlic green tops can be processed further to remove the garlic oil using solvent extraction or supercritical CO2 extraction or chemical extraction methods.

Alternatively, the blended or minced green tops can be stored chilled for short periods of time before further processing or stored frozen for long periods.

The green top blend that has been distilled to remove the garlic oil (spent green tops), is often very warm to hot and is likely to be pasteurised as a result of the distillation process as it may reach nearly 100 degrees Celsius during the process. Once collected, it contains a high level of Brix/sugar, dependent on the maturity of the green top garlic but often around 30 to 40 Brix.

The spent garlic green tops remaining after garlic oil removal can undergo fermentation, whereas previously, the garlic oil would not allow fermentation. However, the solids in the spent garlic green tops need to be separated from the liquid and this can be done by several means including high speed centrifugation, diatomaceous earth filtration combined with rotary drum vacuum filter, cross filtration and other ways known to the expert in the field in removing or separating liquids from solids.

Once solids have been removed, the liquid can be diluted with potable water and the Brix adjusted to between 20 and 25 and the nutritional requirements such as yeast available nitrogen, vitamins, etc. optimised for fermentation and inoculated with alcohol forming yeast suitably selected to ferment this type of sugar solution. The fermenting sugars are converted to ethanol by appropriate yeasts. Non-fermentable sugars can be converted enzymatically prior to initiating fermentation in order to obtain as much ethanol as possible.

Upon completion of the ferment, the alcohol can be distilled or removed by any other known means and used for other products or beverages or fuel etc.

The solids from the spent garlic greens can be used either as a filler in packaged garlic paste products or used as worm feed or other agricultural purposes such as fertilisers or organic material addition to soil.

The garlic oil can be used for medicinal purposes (capsules for human or animal use) as well as food or edible oil fortification, or it can be reverted to allicin through oxidation and acidification and then used as a precursor for Ajoene manufacturing.

Ajoene manufactured from green tops can be used to fortify edible butter or margarine or edible oils for human and animal consumption. Alternatively, the ajoene can be prepared for medical or a microbiological antagonist and capsulated neat, or in combination with other raw garlic constituents such as garlic oil. A combination of garlic oil and Ajoene in a capsule has advantages of acting as an acute antimicrobial agent (garlic oil) as well as preventing expression of important quorum sensing controlled virulence factors, particularly in biofilm forming bacteria (Ajoene).

The main advantage of the present invention is that the green shoots or leaves from the garlic plant can now be used commercially in the production of either Ajoene or garlic oil. Garlic oil from either the garlic bulb or the green shoots or leaves can be oxidised and acidified as described by others to convert it back to allicin, that can then be used as a precursor for making Ajoene.

Further advantages of using an otherwise unused portion of the garlic plant are as follows.

Ajoene produced from green garlic stems that would otherwise be thrown away, reduces the cost of production of both Ajoene and or garlic oil. This will overcome the prohibitive cost of producing Ajoene from a natural source since the green leaves are not as expensive as the garlic bulb. This will allow Ajoene to be accepted as a health promoting compound quicker than a synthetic equivalent Ajoene.

Garlic oil produced from green garlic stems or leaves or shoots is many folds cheaper as the raw source material than garlic bulbs. The garlic oil, in addition to being used as a health supplement in capsules, can also be further processed (oxidation and acidification) to produce Ajoene. It is a simple process to distil the green tops and obtain garlic oil and then use the garlic oil as a source to make ajoene. This saves processing space requirements.

Using the green tops increases the efficiency of garlic production and provides quantitatively more usable material from the same garlic plant than ever before.

The use of the fermentable sugars remaining after removal of garlic oil from garlic green tops offers the opportunity to produce ethanol by fermentation. This reduces environmental impact of garlic processing. The residue from the ferment can be consumed by animals as feed.

In a further aspect of the invention garlic green tops containing allicin can be used for making Ajoene rich butter or margarine, or an edible oil rich in Ajoene for human or animal consumption.

This involves garlic green tops being macerated finely so as to allow the alliin to convert to allicin through enzymes found around the garlic greens. This may involve adding some potable water to make the mixture flowable or pumpable. The solids in such a mixture are then removed physically using any method of separating solids from liquids. The liquid is then collected and either butter cream or edible oil is added to the mixture and the pH is adjusted from between pH 6 and 7 with natural acids such as malic or lactic acid while being mixed. The mixture is incubated at room temperature for at least two hours.

The butter cream or oil are then separated from the liquid by allowing them to physically separate by gravity or by centrifugation and this is removed, to be added to either another butter cream being made into butter or to fresh edible oil. Edible oil rich in Ajoene can also be added to butter or margarine to enrich it with Ajoene.

The reader will now appreciate the present invention which provides a method of harvesting green garlic tops to produce a variety of garlic products from a material that would otherwise be discarded.

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. A method of producing ajoene and garlic oil products from garlic green tops comprising:
harvesting garlic plants and cutting the green tops from garlic bulbs at the time of harvesting;
collecting the green tops in holding bins; and
transporting the green tops to a processing site wherein the green tops are macerated to produce a green top blend and reduce their transportation volume.

2. The method as in claim 1, wherein water is added to the green top blend to make it flowable.

3. The method as in claim 2, wherein cell disruptive enzymes are added to the green top blend to release garlic enzymes that convert alliin to allicin, release garlic oil or convert sugars into fermentable sugars to produce alcohol or vinegar after fermentation.

4. The method of claim 3 wherein distillation or supercritical CO2 is used to separate the garlic oil from the green top blend and leave spent green tops, and wherein liquid is extracted from the spent green tops and fermented to produce alcohol.

5. The method as in claim 3, wherein the allicin is extracted from the green top blend and converted into ajoene.
